(19) United States
(12) Patent Application Publication
(10) Pub. No.: US 2024/0254450 A1
(43) Pub. Date: Aug. 1, 2024

(54) ALVEOLAR ORGANOID GENERATION USING ONLY DECM HYDROGELS WITH DROPPING SUSPENSION TECHNOLOGY

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Ayoung JO, Seoul (KR); Min Jae LIM, Gyeonggi-do (KR)

(21) Appl. No.: 18/427,981

(22) Filed: Jan. 31, 2024

(30) Foreign Application Priority Data
Jan. 31, 2023 (KR) 10-2023-0013343
Jan. 26, 2024 (KR) 10-2024-0012658

(51) Int. Cl. C12N 5/071; A61K 35/42

(52) U.S. Cl. CPC C12N 5/0688; A61K 35/42; C12N 2533/90

(57) ABSTRACT

The present invention relates to a method of producing an alveolar organoid by a dropping suspension culture technique using decellularized extracellular matrix (dECM) hydrogel, and according to the present invention, when an organoid is produced by a dropping suspension culture method using dECM, as in a living organism, an organoid in which cells and dECM are fused may be formed. Moreover, since dECM is what remains after all cells have been removed from tissue and thus is easily applied clinically, the produced organoid is expected to be useful for disease modeling, drug screening, regenerative medicine, etc.

### TECHNICAL FIELD

The present invention relates to a method of producing an alveolar organoid using a dECM hydrogel to which a dripping suspension culture technique is applied.

### BACKGROUND ART

Currently, animal models are widely used as disease models. However, since animals are physically different from humans, there are limitations in replicating human diseases in animals, and additionally, ethical issues caused by these practices are constantly emerging.

Therefore, organoids, which are three-dimensional cell structures, have been recently attracting attention because they consist of various cells that constitute tissue and can mimic the in vivo environment, so they are widely studied and applied in various areas, from basic biology research to various applied research fields such as new drug development, disease modeling, and regenerative therapy. In addition, organoids are attracting more attention because the use of organoid models produced from patient-derived cells enables research on disease mechanisms and even customized diagnosis of patients.

Meanwhile, various types of organoid models derived from stem cells of various organs in the living organism have been constructed. To culture these various types of organoids, many researchers in the world use Matrigel as a culture scaffold. However, because Matrigel is an ingredient derived from mouse sarcoma, concerns have been raised about attempts to transplant organoids cultured in Matrigel into humans due to the risk of injection and immune rejection. In addition, because Matrigel is not a tissue-specific decellularized extracellular matrix ingredient, it cannot provide an optimized microenvironment for organoid differentiation.

In addition, lung diseases, which are caused by genetic factors, excessive smoking, fine dust generated from air pollution, viruses, or bacteria, are one of the major causes of death worldwide. However, due to the lack of a disease model, it is not easy to study the mechanism of a lung disease and the development of a treatment method is also very difficult. Accordingly, there is an urgent need to develop an in vitro model of a lung disease that can precisely mimic a human lung disease.

### RELATED ART DOCUMENTS

### [Patent Document]

(Patent Document 001) Korean Patent Document No. 10-1135709

### DISCLOSURE

### Technical Problem

As a result of conducting extensive research for producing an alveolar organoid similar to a living organism, the present inventors confirmed that, when an alveolar organoid is produced by dropping suspension culture using a decellularized extracellular matrix (dECM) instead of Matrigel, which is difficult to apply clinically, unlike a conventional culture method in which multiple heterogeneous organoids composed of only cells were produced, a single organoid composed of cells and a decellularized extracellular matrix is produced uniformly, and thus the present invention was completed.

Therefore, the present invention is directed to providing a method of producing an alveolar organoid using only dECM.

The present invention is also directed to providing an alveolar organoid produced by the method according to the present invention.

The present invention is also directed to providing a pharmaceutical composition for preventing or treating a respiratory disease, which includes the alveolar organoid according to the present invention as an active ingredient.

However, technical problems to be solved in the present invention are not limited to the above-described problems, and other problems which are not described herein will be fully understood by those of ordinary skill in the art from the following descriptions.

### Technical Solution

To achieve the above purposes, the present invention provides a method of producing an alveolar organoid using dECM, which includes: (S1) preparing a bioink by mixing a first medium, type 2 alveolar cells, and a decellularized extracellular matrix (dECM);
(S2) forming a cell culture droplet by dispensing the bioink into a plate;
(S3) gelating the cell culture droplet; and
(S4) isolating the gelated cell culture droplet from the plate and culturing it in a second medium.

In one embodiment of the present invention, the method may not use Matrigel, but the present invention is not limited thereto.

In another embodiment of the present invention, the alveolar organoid may be cultured in a non-adherent manner, but the present invention is not limited thereto.

In still another embodiment of the present invention, the first medium may be a small airway epithelial cell growth medium (SAGM), but the present invention is not limited thereto.

In yet another embodiment of the present invention, the second medium may be an alveolarization medium, but the present invention is not limited thereto.

In yet another embodiment of the present invention, the alveolarization medium may include one or more selected from the group consisting of Ham's F12, dexamethasone, 3-isobutyl-1-methylxanthine (IBMX), B27 supplement, bovine serum albumin (BSA), hydroxyethyl piperazine ethane sulfonic acid (HEPES), calcium chloride, ITS premix, 8-Br-cAMP, fibroblast growth factor 7 (FGF7), and penicillin/streptomycin, but the present invention is not limited thereto.

In yet another embodiment of the present invention, the dECM may be prepared by a method including the following steps, but the present invention is not limited thereto:
a) cutting lung tissue isolated from a subject to a size of 0.01 to 100 mm in diameter;
b) decellularizing the tissue cut in a);
c) removing fat from the decellularized tissue;
d) removing genetic material from the defatted tissue;
e) enzymatically digesting the genetic material-removed tissue with pepsin; and
f) neutralizing the enzymatically digested tissue.

In yet another embodiment of the present invention, the decellularization may be performed by the following steps, but the present invention is not limited thereto:
b-1) treatment with sodium dodecyl sulfate (SDS);
b-2) treatment with Triton X-100; and
b-3) washing.

In yet another embodiment of the present invention, the method may further include g) performing gelation, but the present invention is not limited thereto.

In yet another embodiment of the present invention, the dECM may include one or more selected from the group consisting of the proteins listed in Table 2 below, but the present invention is not limited thereto.

In yet another embodiment of the present invention, the bioink may include the dECM at a concentration of 0.01 to 10% (w/v), but the present invention is not limited thereto.

In addition, the present invention provides an alveolar organoid produced by the method according to the present invention.

In one embodiment of the present invention, the alveolar organoid may be a form in which the dECM and alveolar cells are fused, but the present invention is not limited thereto.

In addition, the present invention provides a pharmaceutical composition for preventing or treating a respiratory disease, which includes the alveolar organoid according to the present invention as an active ingredient.

In addition, the present invention provides a method of preventing or treating a respiratory disease, comprising: administering a composition comprising the alveolar organoid according to the present invention to a subject in need thereof.

In addition, the present invention provides a use of a composition comprising the alveolar organoid according to the present invention for preventing or treating a respiratory disease.

In addition, the present invention provides a use of the alveolar organoid according to the present invention for preparing a therapeutic agent for a respiratory disease.

### Advantageous Effects

According to the present invention, when an organoid is produced by a dropping suspension culture method using a decellularized extracellular matrix (dECM), as in an organism, an organoid can be formed such that cells are fused with dECM. Moreover, since dECM is what remains after all cells have been removed from tissue and thus is easy to be applied clinically, the produced organoid is expected to be useful in disease modeling, drug screening, and regenerative medicine, etc.

### DESCRIPTION OF DRAWINGS

FIG. 1 is a schematic diagram that illustrates a dropping suspension culture method according to one embodiment of the present invention.

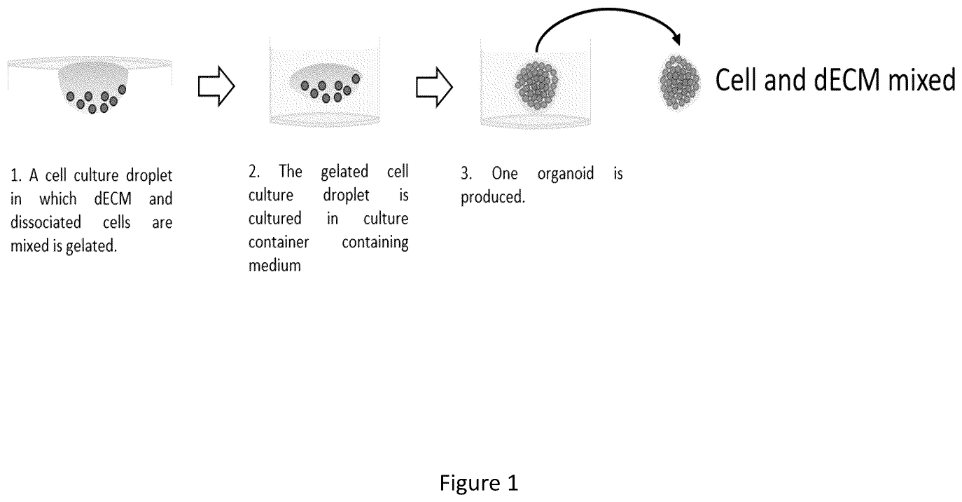

FIG. 2 is a schematic diagram that shows the difference between dropping suspension culture using Matrigel, hanging drop culture and dome culture, and dropping suspension culture using dECM according to one embodiment of the present invention.

FIG. 3 is a view that shows the results of confirming morphologies of organoids produced by dropping suspension culture using dECM and dropping suspension culture using Matrigel.

FIG. 4 is a view that shows the results of confirming the sizes of organoids produced by dropping suspension culture using dECM and dropping suspension culture using Matrigel.

FIG. 5 is a view that shows the results of confirming the growth process of an organoid produced by dropping suspension culture using dECM.

FIG. 6 is a view that shows the cross-section of an organoid produced by dropping suspension culture using dECM.

### MODES OF THE INVENTION

As a result of conducting extensive research for producing an alveolar organoid as in a living organism, the present inventors confirmed that, when an alveolar organoid is produced by dropping suspension culture using a decellularized extracellular matrix (dECM), rather than Matrigel, which is difficult to apply clinically, compared to a conventional culture method in which multiple heterogeneous organoids composed of only cells were produced, a single organoid formed of cells and dECM is produced uniformly. Thus, the present invention was completed.

Hereinafter, the present invention will be described in detail.

The present invention provides a method of producing an alveolar organoid using dECM, which includes (S1) preparing a bioink by mixing a first medium, type 2 alveolar cells, and a decellularized extracellular matrix (dECM);
(S2) forming a cell culture droplet by dispensing the bioink into a plate;
(S3) gelating the cell culture droplet; and
(S4) isolating the gelated cell culture droplet from the plate and culturing it in a second medium, and particularly, the alveolar organoid production method provides an alveolar organoid by dropping suspension culture.

The term “organoid production (culture)” used herein includes all actions that can produce or maintain organoids. For example, this may include allowing cells or cells isolated from specific tissue to differentiate into tissue or organ cells with a specific function, and/or allowing organoids to survive, grow, or proliferate.

The term “dropping suspension culture” used herein refers to a new culture method developed by improving the hanging drop culture method, and refers to a method of forming an organoid, which includes forming a cell culture droplet by hanging a drop of a mixture of cells and hydrogel, gelating the cell culture droplet, isolating the gelated cell culture droplet and putting it into a culture container, and then adding a medium to submerge the cell culture droplet for incubation. The dropping suspension culture refers to a culturing method of forming an organoid by locating the cell in the hydrogel in the process of gelating the cell culture droplet, that is, the cell-mixed hydrogel, and culturing the gelated cell culture droplet in a non-adherent form.

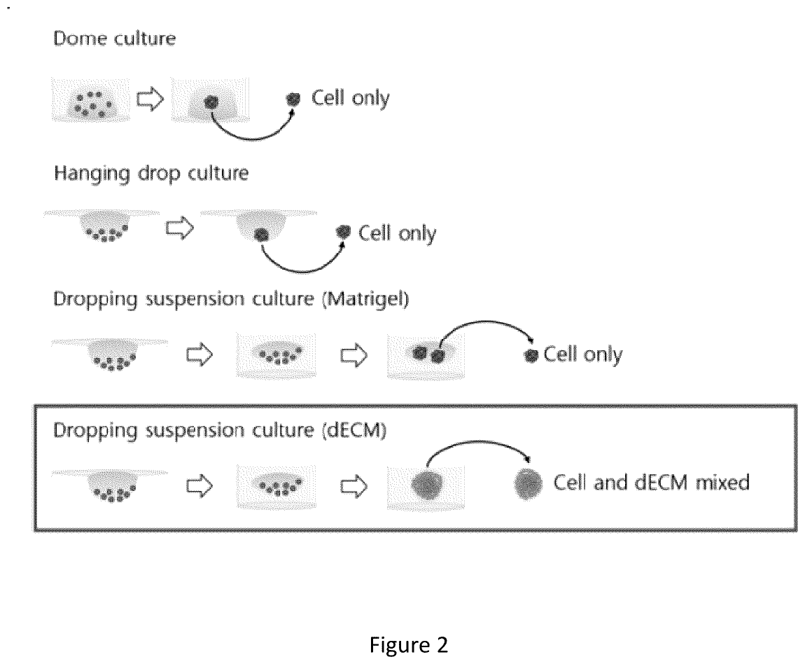

An alveolar organoid produced by the alveolar organoid production method according to the present invention is characterized by being cultured by dropping suspension culture, that is, non-adherent culture. As shown in FIG. 2, when being cultured through non-adherent culture, since cells are cultured in hydrogel without adhering to a culture dish or lid, uniform organoids can be produced unlike conventional dome culture or hanging drop culture in which cells differentiate non-uniformly due to the differences between cells on an adherent surface and cells on a non-adherent surface.

The term “non-adherent culture” used herein refers to a method of culturing cells or organoids by suspending them in a medium without adhering to the bottom of a culture dish. Compared with an adherent culture method, this method does not need a detachment process using a chemical decomposition enzyme such as trypsin, maintains cells mainly by diluting cultured cells, or simply increases the amount of medium without subculture. This allows cells to be cultured continuously. In non-adherent culture, if cell concentration can be maintained constantly through continuous dilution of a culture solution, cell proliferation can reach a steady state, which is not easily achieved in adherent cell culture.

According to one embodiment of the present invention, the first medium is a small airway epithelial cell growth medium (SAGM), and the second medium may be an alveolarization medium shown in Table 1 below, but the present invention is not limited thereto.

The term “medium” used herein refers to a composition that includes the nutrients required to maintain the growth and survival of cells or organoids in vitro.

The phrase “small airway epithelial cell growth medium” or “SAGM” refers to a growth medium containing one or more of the following ingredients, such as hydrocortisone, epidermal growth factor, epinephrine, transferrin, insulin, retinoic acid, triiodothyronine, and fatty acid-free BSA (bovine serum albumin).

The term “alveolarization medium” used herein may be a medium that enables the culture, growth, and/or proliferation of type 2 alveolar cells into alveolar organoids, but the present invention is not limited thereto. According to one embodiment of the present invention, before being cultured in the alveolarization medium, the cells may be cultured in SAGM for one week, and then cultured in an alveolarization medium.

The alveolarization medium may include one or more selected from the group consisting of Ham's F12, dexamethasone, 3-isobutyl-1-methylxanthine (IBMX), B27 supplement, bovine serum albumin (BSA), hydroxyethyl piperazine ethane sulfonic acid (HEPES), calcium chloride, ITS premix, 8-Br-cAMP, fibroblast growth factor 7 (FGF7), and penicillin/streptomycin, but the present invention is not limited thereto.

The alveolarization medium may include 50 to 150 μM IBMX, B27 supplement, 20 to 70 nM dexamethasone, 0.01 to 1% ITS premix, 0.1 to 0.5% BSA, 0.5 to 1.5 mM calcium chloride, 10 to 20 mM HEPES, 10 to 200 μM 8-Br-cAMP, 1 to 20 ng/mL FGF7, and/or 10 to 100 U/mL penicillin/streptomycin with respect to 20 mL of Ham's F12, but the present invention is not limited thereto.

The alveolarization medium may include, but is not limited to, one or more selected from the group consisting of 8-Br-cAMP and FGF7 before use.

According to one embodiment of the present invention, as a result of preparing organoids through the dropping suspension culture using Matrigel or dECM, in the case of organoids cultured in Matrigel, several organoids consisting of only cells with non-uniform sizes were produced, whereas in the case of organoids cultured in dECM, one organoid in which cells and dECM were fused (coexist) as in a living organism was formed, confirming that it was appropriate that dECM is used as hydrogel through the dropping suspension culture. Therefore, the method of producing an alveolar organoid according to the present invention does not use Matrigel.

While Matrigel is derived from tumor cells and thus has limitations in clinical application and a separate process of removing Matrigel is required to clinically apply organoids cultured in Matrigel, dECM has an advantage that the prepared living organism-derived organoids can be clinically applied without a separate removal process.

The “extracellular matrix (ECM)” used herein is a matrix that surrounds the outside of cells and has a network structure that occupies the space between cells and is mainly composed of proteins and polysaccharides. Components of ECM include structural components such as collagen and elastin, adhesive proteins such as fibronectin, vitronectin, laminin, and tenascin, chondroitin sulfate or heparan sulfate, proteoglycans produced from main proteins, and hyaluronic acid consisting of only polysaccharides. These ECM components are key materials that not only determine the morphology of the tissue that cells combine to form, but also provide an environment in which cells can function normally and are involved in cell differentiation. According to one embodiment of the present invention, in the present invention, the ECM may be derived from a pig, but the present invention is not limited thereto.

The decellularization process is a technology that isolates dECM components contained in biological tissue through chemical treatment. dECM components also include a large number of collagens obtained through the decellularization process and various proteins involved in the composition and function implementation of the corresponding biological tissue, and these components are expected to provide a more specialized biological environment for artificial organ regeneration.

In the present invention, the dECM may be prepared by the following steps, but the present invention is not limited thereto:
a) cutting lung tissue isolated from a subject to a size of 0.01 to 100 mm in diameter;
b) decellularizing the tissue cut in a);
c) removing fat from the decellularized tissue;
d) removing genetic material from the defatted tissue;
e) enzymatically digesting the genetic material-removed tissue with pepsin; and
f) neutralizing the enzymatically digested tissue.

In the present invention, the decellularization step may include the following steps, but the present invention is not limited thereto:
b-1) treatment with sodium dodecyl sulfate (SDS);
b-2) treatment with Triton X-100; and
b-3) washing.

In the present invention, the decellularization may be performed by a method known in the art, and in one embodiment of the present invention, the lung tissue obtained from a tissue source was washed with deionized water and PBS, washed sequentially with isopropanol, PBS, and deoxyribonuclease, enzymatically digested with pepsin, and then neutralized.

In the present invention, the fat removal in c) may be performed using isopropanol, but the present invention is not limited thereto. The isopropanol may be treated for 10 to 48 hours, 10 to 40 hours, 10 to 32 hours, 14 to 32 hours, 18 to 28 hours, 20 to 28 hours, 22 to 26 hours, 23 to 25 hours, or approximately 24 hours, but the present invention is not limited thereto.

In the present invention, the removal of genetic material in d) may be performed using DNase, but the present invention is not particularly limited thereto.

In the present invention, the pepsin in e) may be treated at 1 to 40% (w/w), 1 to 30% (w/w), 5 to 30% (w/w), 10 to 30% (w/w), 15 to 25% (w/w), or approximately 20% (w/w) relative to the dECM concentration, but the present invention is not limited thereto.

In the present invention, the neutralization in f) may be performed with one or more selected from the group consisting of NaOH and PBS, but the present invention is not limited thereto.

In the present invention, the method may further include g) performing gelation, but the present invention is not limited thereto.

In the present invention, the lung tissue in a) may be cut to a size of 0.01 to 100 mm, 0.01 to 50 mm, 0.01 to 40 mm, 0.01 to 30 mm, 0.01 to 20 mm, 0.01 to 10 mm, 0.01 to 5 mm, 0.01 to 3 mm, 0.01 to 2 mm, or 0.1 to 2 mm, but the present invention is not limited thereto.

In the present invention, the bioink may include dECM at 0.01 to 10% (w/v), 0.01 to 9% (w/v), 0.01 to 8% (w/v), 0.01 to 7% (w/v), 0.01 to 6% (w/v), 0.01 to 5% (w/v), 0.01 to 4% (w/v), 0.01 to 3% (w/v), 0.01 to 2% (w/v), 0.01 to 1% (w/v), 0.01 to 0.5% (w/v), 0.05 to 0.5% (w/v), or approximately 0.06 to 0.12% (w/v), but the present invention is not limited thereto.

In the present invention, 1×10⁴ to 1×10⁵, 1×10⁴ to 9×10⁴, 1×10⁴ to 8×10⁴, 1×10⁴ to 7×10⁴, 1×10⁴ to 6×10⁴, 1×10⁴ to 5×10⁴, 4×10⁴ to 6×10⁴, or approximately 5×10⁴ type 2 alveolar cells may be included per 50 μL of dECM, but the present invention is not limited thereto.

In another embodiment of the present invention, the dECM may include one or more selected from the group consisting of proteins listed in Table 2 below, but the present invention is not limited thereto.

In addition, the present invention provides an alveolar organoid prepared by the method according to the present invention.

In the present invention, the alveolar organoid may be a form in which the dECM and alveolar cells are fused.

According to one embodiment of the present invention, when organoids are prepared through dropping suspension culture using dECM, the prepared organoids may be prepared to have a uniform size and are characterized by excellent repeatability. Accordingly, it is easy to mass produce the alveolar organoid according to the present invention. Specifically, the alveolar organoids prepared by the method according to the present invention may have a size variation between organoids of 1 to 50%, 1 to 45%, 1 to 40%, 1 to 35%, 1 to 30%, 1 to 25%, 1 to 20%, 1 to 15%, 1 to 10%, 1 to 5%, 5 to 50%, 5 to 45%, 5 to 40%, 5 to 35%, 5 to 30%, 5 to 25%, 5 to 20%, 5 to 15%, or 5 to 10%.

Since the alveolar organoid of the present invention may be used in treatment in vivo, the present invention may provide a pharmaceutical composition for preventing or treating a respiratory disease, which includes the alveolar organoid as an active ingredient.

In addition, the present invention provides a method of preventing or treating a respiratory disease, comprising: administering a composition comprising the alveolar organoid according to the present invention to a subject in need thereof.

In the present invention, the prevention or treatment method may further include administering the alveolar organoid or transplanting the alveolar organoid, and as a result of administration or transplantation, the symptoms of a respiratory disease in a patient are improved, treated, or alleviated. The improvement, treatment or alleviation may be any changes in the respiratory disease or symptoms of the respiratory disease, which can be detected using natural senses or artificial devices.

In addition, the present invention provides a use of a composition comprising the alveolar organoid according to the present invention for preventing or treating a respiratory disease.

In addition, the present invention provides a use of the alveolar organoid according to the present invention for preparing a therapeutic agent for a respiratory disease.

The “patient” used herein may be a human or a non-human mammal. Non-human mammals include, for example, livestock and pets, such as sheep, cattle, pigs, dogs, cats, and murine animals.

Respiratory diseases that can be treated by the method of the present invention include diseases or conditions physically manifested in the respiratory tract, and examples include, but are not limited to, cystic fibrosis, respiratory distress syndrome, acute respiratory distress syndrome, pulmonary tuberculosis, coughs, bronchial asthma, coughs based on increased airway hyperresponsiveness (bronchitis, flu syndrome, asthma, obstructive pulmonary disease, etc.), flu syndrome, cough suppression, airway hypersensitivity, tuberculosis disease, asthma (airway inflammatory cell infiltration, increased airway hyperresponsiveness, bronchoconstriction, mucus hypersecretion, etc.), chronic obstructive pulmonary disease, emphysema, pulmonary fibrosis, idiopathic pulmonary fibrosis, reversible airway obstruction, adult respiratory disease syndrome, pigeon breeder's disease, farmer's lung, bronchopulmonary dysplasia, airway disease, allergic bronchopulmonary aspergillus, allergic bronchitis bronchiectasis, occupational asthma, reactive airway disease syndrome, intestinal lung disease, or parasitic lung disease.

The alveolar organoid of the present invention may be included at 1 pg to 30 g w/v % in the pharmaceutical composition, but the present invention is not limited thereto.

The “subject” used herein is not limited as long as it is a vertebrate, and it may specifically be, a human, a mouse, a rat, a guinea pig, a rabbit, a monkey, a pig, a horse, a cow, sheep, an antelope, a dog, or a cat, and preferably, a human or a pig.

The “administration” used herein may refer to the introduction of a pharmaceutical composition of the present invention into a patient by any suitable method, and the administration route of the composition of the present invention may be administered via various oral or parenteral routes as long as it can reach the desired tissue.

The “prevention” used herein refers to all actions that delay a respiratory disease by the administration of the composition according to the present invention, the “treatment” used herein refers to all actions involved in alleviating or beneficially changing the symptoms of a respiratory disease by the administration of the pharmaceutical composition according to the present invention, and the “improvement” used herein refers to all actions that reduce parameters associated with a respiratory disease, such as the severity of symptoms, by the administration of the composition according to the present invention.

In the present invention, the pharmaceutical composition may further include suitable carriers, excipients, and diluents, which are conventionally used in the preparation of pharmaceutical compositions.

The “carrier” used herein, also called a vehicle, refers to a compound that facilitates the addition of a protein or peptide into cells or tissue, and for example, dimethyl sulfoxide (DMSO) is a carrier that is generally used to facilitate the introduction of various organic materials into the cells or tissue of a living organism.

The “diluent” used herein refers to a compound diluted in water that not only stabilizes the biologically active form of a target protein or peptide but also dissolves a protein or peptide. A salt that is dissolved in a buffer solution is used as a diluent in the art. A commonly used buffer solution is phosphate-buffered saline because it mimics the salt state of a human body fluid. Because a buffer salt can control the pH of a solution at low concentrations, it is rare for a buffer diluent to modify the biological activity of a compound. Compounds containing azelaic acid used herein may be administered into a human patient alone, or in combination with other components or an appropriate carrier or excipient as a pharmaceutical composition as in combination therapy.

In addition, the pharmaceutical composition according to the present invention may be used in the form of drugs for external use such as powders, granules, tablets, capsules, suspensions, emulsions, syrups, and aerosols, or sterile injection solutions according to conventional methods. The carrier, excipient and diluent, which may be included in the composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil. The composition may be formulated with a diluent or an excipient such as a filler, a thickening agent, a binder, a wetting agent, a disintegrant, and a surfactant, which are commonly used. Solid formulations for oral administration may include tablets, pills, powders, granules or capsules, and such solid formulations may be prepared by mixing at least one excipient, for example, starch, calcium carbonate, sucrose, lactose or gelatin, with the active ingredient. Also, in addition to simple excipients, lubricants such as magnesium stearate and talc may also be used. As liquid formulations for oral administration, suspensions, liquids for internal use, emulsions, or syrups may be used, and a generally used simple diluent such as water or liquid paraffin, as well as various types of excipients, for example, a wetting agent, a sweetener, a fragrance and a preservative may be included. Formulations for parenteral administration may include sterilized aqueous solutions, non-aqueous solvents, suspensions, emulsions, freeze-dried formulations, or suppositories. As the non-aqueous solvent or suspension, propylene glycol, polyethylene glycol, a vegetable oil such as olive oil, or an injectable ester such as ethyl oleate may be used. As a suppository base, Witepsol, Tween 61, cocoa butter, laurin fat, glycerol, or gelatin may be used.

The pharmaceutical composition of the present invention may be orally or parenterally, and preferably, parenterally administered, and for parenteral administration, the pharmaceutical composition of the present invention may be administered via muscular injection, intravenous injection, subcutaneous injection, intraperitoneal injection, topical administration, or transdermal administration. The appropriate dosage of the pharmaceutical composition of the present invention may be prescribed in various ways according to factors such as a formulation method, an administration method, patient's age, body weight, sex, pathogenic condition, diet, administration time, administration route, excretion rate, and reaction sensitivity. The pharmaceutical composition of the present invention may be prepared in a unit dose form or in a large-capacity container by formulating it using pharmaceutically acceptable carrier and/or excipient according to a method that can be easily performed by one of ordinary skill in the art to which the present invention belongs. Here, the formulation of the pharmaceutical composition of the present invention may be in the form of a solution, suspension or emulsion in an oil or aqueous medium, an elixir, a powder, a granule, a tablet, or a capsule, and may further include a dispersant or a stabilizer.

The terms used in the present invention have been selected from general terms that are currently and widely used as much as possible while considering the functions of the present invention, but this may vary depending on the intentions of technicians skilled in the art, precedents, and the emergence of new technologies. In addition, in certain cases, there are also terms arbitrarily selected by the applicants, and in this case, their meanings will be described in detail in the description of the corresponding invention. Therefore, the terms used in the present invention should be defined based on the meanings of the terms and the content throughout the present invention, not simply based on the names of the terms used in the present invention.

The ordinal numbers, for example, “first” and “second,” may be used to describe various components, but the components should not be limited by these terms. The terms are used only to distinguish one component from another component. For example, without departing from the scope of the embodiments, a second component may be referred to as a first component, and similarly, the first component may be referred to as a second component. The term “and/or” encompasses a combination of a plurality of related stated items or any one of a plurality of related stated items.

Throughout the specification, when one part “includes” a component, it means that it may also include other components rather than excluding components unless particularly stated otherwise. The term “approximately” or “substantially” used herein are used at, or in proximity to, numerical values when allowable manufacturing and material tolerances, which are inherent in the meanings, are presented. This term is used to prevent the unfair use of the disclosure in which correct or absolute values are cited to help in understanding the present invention by unscrupulous infringers.

Throughout the specification, the term “combination thereof” included in the Markush-type expression refers to a mixture or combination of one or more selected from the group consisting of constituents described in the Markush-type expression, that is, one or more selected from the group consisting of the components.

Hereinafter, preferred examples are presented to better understand the present invention. However, the following examples are provided to more easily understand the present invention, and the content of the present invention is not limited by the following examples.

### Experimental Method

1. Isolation and 2D Culture of Type 2 Alveolar Cells from Lung Tissue

Human lung tissue was obtained during lung surgery and used with the patient's content before surgery. Normal lung tissue was collected, washed with HBSS 2 to 3 times, cut into pieces having a final size of approximately 0.2 to 0.5 mm, and then the blood in the tissue was removed. The tissue was put in a shaking incubator at 37° C. for 45 minutes along with a lysis buffer (trypsin, elastase with HBSS). After filtering large tissue using a 70-μm strainer, centrifugation was performed at 1500 rpm for 10 minutes. Cells resuspended in PBS were filtered using a 40-μm strainer and centrifuged at 1000 rpm for 1 to 2 minutes to remove red blood cells (RBCs). After removing RBCs, the cells centrifuged at 1500 rpm for 5 minutes were resuspended in SAGM, and then seeded on a Matrigel- or dECM-coated plate or an uncoated dish. For the seeded cells, SAGM was changed once every 2 to 3 days.

2. Construction of dECM from Lung Tissue

Frozen lung tissue of a pig stored at −80° C. was sliced into 1 mm-thick pieces. The sliced tissue was washed with PBS five times. The washed tissue was decellularized by the following steps:

\textcircled{1} SDS in PBS for 2 days (replaced with SDS buffer once every 24 hours), \textcircled{2} Triton X-100 in PBS for 2 days (replaced once every 24 hours), and \textcircled{3} washed with PBS overnight. Decellularized lung tissue, i.e., dECM, was washed with isopropanol for 24 hours to remove fat. Subsequently, the resulting product was washed with PBS 4 times for one hour each, treated with DNase, and washed with PBS four times for one hour each. The washed dECM was stored at −80° C. and lyophilized. The lyophilized dECM was subjected to enzymatic digestion (solubilization) using pepsin (pH 2.5) in 0.5 M acetic acid. Here, the pepsin concentration was 20% (w/w) of the dECM concentration, and the amount of dECM was adjusted such that the final concentration of a hydrogel stock solution was 1% (w/v). The enzymatically digested dECM was neutralized in 10M NaOH, 10×PBS at pH 7.3 to 7.5. After neutralization, the temperature was adjusted to 37° C., and the dECM hydrogel stock solution was prepared through gelation by incubation for 1 hour.

### 3. 3D Organoid Culture Using Dropping Suspension Culture

Type 2 alveolar cells isolated from human lung tissue were seeded in a mixture of Corning Matrigel, collagen-based hydrogel, and SAGM in a 6:4 ratio. dECM was mixed with SAGM such that the concentration of a 1% (w/v) dECM hydrogel stock solution was 0.06 to 0.12% and then dispensed. The type 2 alveolar cells were seeded at 5000 cells/50 μL of hydrogel and gently pipetted to mix the SAGM, the type 2 alveolar cells and the hydrogel well, thereby preparing a bioink. The bioink in which the cells and hydrogel was mixed was dispensed into a 24-well Cryschem plate (Hampton Research, CAT #HR3-159) to form a cell culture droplet as in hanging-drop culture, and then gelated in a 37° C. incubator for 1 to 2 hours. The gelated cell culture droplet (bioink gel mass) was detached from the 24-well Cryschem plate and put into a 6-well SPL3D™ Cell Floater (SPL, cat #39706). Subsequently, SAGM was added to submerge the cell culture droplet and cultured in a 37° C. incubator for 7 days and replaced with an alveolarization medium for incubation. The composition of the alveolarization medium is as follows: dexamethasone, 3-isobutyl-1-methylxanthine (IBMX), B27 supplement, bovine serum albumin (BSA), hydroxyethyl piperazine ethane sulfonic acid (HEPES), calcium chloride, ITS premix, 8-Br-cAMP, fibroblast growth factor 7 (FGF7), and penicillin/streptomycin in Ham's F12 medium (however, 8-Br-cAMP and FGF7 were added before use).

TABLE 1

| Media component | Final concentration | |
|---|---|---|
| 8-Br-CAMP | 100 | uM |
| FGF7 (human KGF) | 10 | ng/ml |
| IBMX | 100 | uM |
| B27 supplement | 1x | |
| Dexamethasone | 50 | nM |
| ITS premix | 0.10% | |
| BSA | 0.25% | |
| CaCl₂ | 0.8 | mM |
| Hepes | 15 | mM |
| Penicillin/Streptomycin | 100 U · ml⁻¹/100 v g · ml⁻¹ | |
| Ham's F12 | 20 | ml |

A dropping suspension culture method is as follows:
1) Cells were isolated from the culture medium.
2) The number of isolated cells was counted, and the cells were transferred to a 1.5-mL tube.
3) SAGM and hydrogel were put into the 1.5-mL tube containing the cells at a volume of 1×10⁵/mL.
4) A bioink was prepared by gently pipetting to mix the SAGM, the cells, and the hydrogel well.
5) The bioink in which the cells and the hydrogel were mixed was dispensed into a 24-well Cryschem Plate (Hampton Research, CAT #HR3-159) to form a cell culture droplet as in hanging drop culture.
6) The cell culture droplet was gelated in a 37° C. incubator for 1 to 2 hours.
7) The gelated cell culture droplet (bioink gel mass) was detached from the 24-well Cryschem plate and added to a 6-well SPL3D™Cell Floater (SPL, cat #39706).
8) The resulting product was put into SAGM to submerge the cell culture droplet and cultured in a 37° C. incubator for 7 days, and then the medium was replaced with an alveolar medium (AM) for incubation.

### 4. Immunofluorescence Staining

After fixing an organoid dome being cultured with 4% PFA, the Catholic University Joint Research Support Center was requested to produce a paraffin block, and a tissue section slide was produced with a thickness of 8 μm. The paraffin in the tissue section slide was removed with xylene and rehydrated with ethanol, and then retrieval was performed using proteinase K solution.

Normal goat serum (NGS) was used to perform blocking, and primary antibodies were diluted in 5% NGS (in PBS), and incubated overnight at 4° C.

The primary antibodies used are as follows: anti-TTF1 antibodies (1:500, Abcam), surfactant protein C antibodies (1:500, Thermo Fisher), recombinant anti-aquaporin 5 antibodies (1:400, abcam), anti HT2-280 antibodies (1:200, Terrace Biotech), anti HT1-56 antibodies (1:200, Terrace Biotech), caveolin-1 antibodies (1:500, abcam), and anti-uteroglobin antibodies (CCSP)(1:400, abcam). Secondary antibodies were diluted in 5% NGS (in PBS) and incubated at room temperature for 1 hour. Alexa Fluor 488 recombinant polyclonal antibodies (1:1000, Thermo Fisher) and Alexa Fluor 546 recombinant polyclonal antibodies (1:1000, Thermo Fisher) were used as secondary antibodies. The degree of fluorescence expression was measured using a confocal laser scanning microscope (LSM800 w/Airyscan, Carl Zeiss).

### 5. Light Microscopy and DAPI Staining

Axiovert 200 (Zeiss, Oberkochen, Germany) optical microscope equipment was used to take photographs at 100 or 200× magnification.

For DAPI staining, a target was treated with Hoechst 33342 (USA, Invitrogen) in PBS at 1:5000, reacted at ordinary temperature for 10 minutes, and washed with PBS at ordinary temperature for 5 minutes.

### 6. Proteomics Analysis

Proteometech Inc. was requested to conduct proteomics analysis.

### EXAMPLES

### Example 1. Comparison of Dropping Suspension Culture in Matrigel and dECM

### 1-1. Comparison in Morphology of Organoids

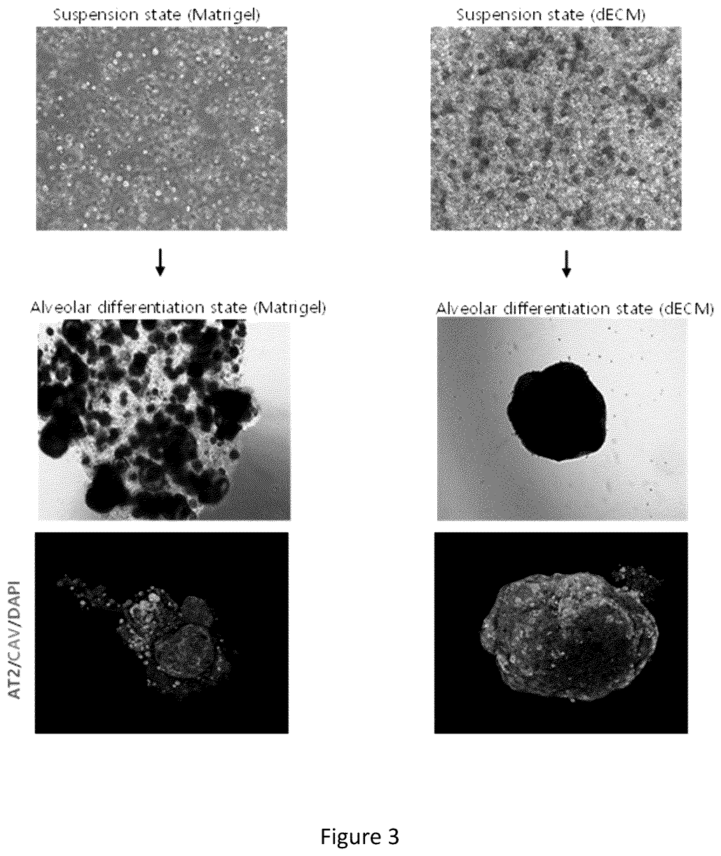

The morphologies of organoids produced according to the type of hydrogel in dropping suspension culture were compared. As a result, as shown in FIG. 3, when cells were cultured in Matrigel, it was confirmed that several organoids with various sizes were produced, which is the same as in a conventionally used dome-shaped organoid culture method, which produces several organoids in one well. Conversely, when cells were cultured in dECM by a dropping suspension culture technique, it was confirmed that, unlike the conventional method, they were cultured in a non-adherent manner, and dECM and the cells were agglomerated into one organoid and grown.

### 1-2. Comparison of Organoid Size

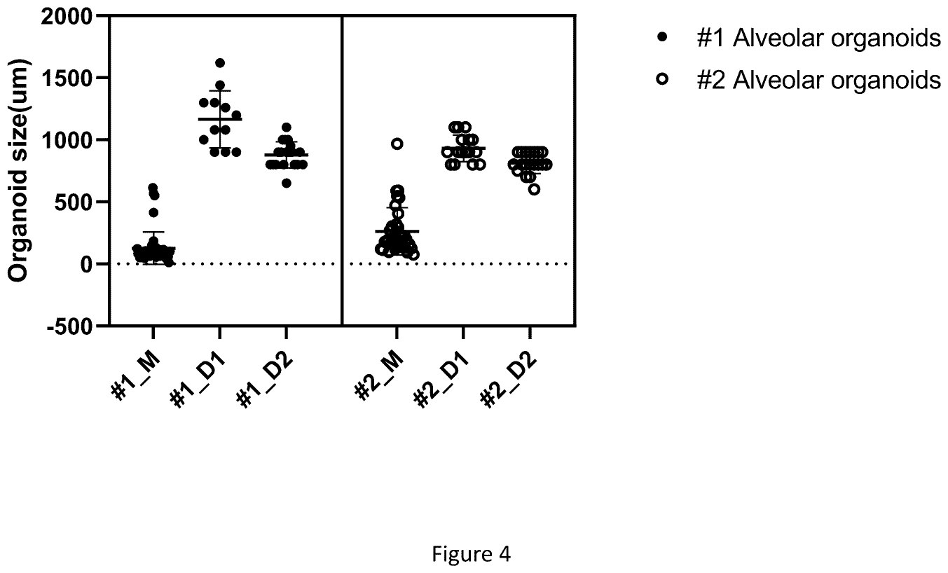

The sizes of organoids produced in Matrigel (M) and dECM (D), derived from different people (#1 and #2), were compared. As a result, as shown in FIG. 4, it was observed that the sizes of organoids cultured in Matrigel were #1_M: 51-610 μm and #2_M: 76-960 μm, and the sizes of D1 and D2 organoids cultured in dECM with different concentrations were #1 D1: 900-1620 μm, #1_D2: 650-1100 μm, #2_D1: 800-1100 μm, and #2_D2: 600-900 μm, indicating that the organoids were grown consistently to similar sizes depending on concentration.

As shown in FIG. 4, the organoids produced in Matrigel are produced in a variety of sizes from 100 μm or less to 500 μm or more, whereas the size variation of the organoids produced in dECM is smaller than that of the organoids produced in Matrigel, inferring that there is size repeatability.

On the other hand, when organoids are prepared in Matrigel, several small organoids are formed, indicating that the organoids are uniformly produced. However, since small organoids are less useful not only in clinical trials but also in experiments, it was determined that, in the case of dropping suspension culture, it is appropriate to use dECM, which has size repeatability and uniformly produces large organoids.

Therefore, from the above results, it was determined that production of uniform organoids would be possible due to reproducibility and size repeatability when producing organoids by dropping suspension culture using dECM.

### Example 2. Characterization of Organoids Produced by Dropping Suspension Culture Using dECM

Organoids produced by dropping suspension culture using dECM were observed using light microscopy and DAPI staining.

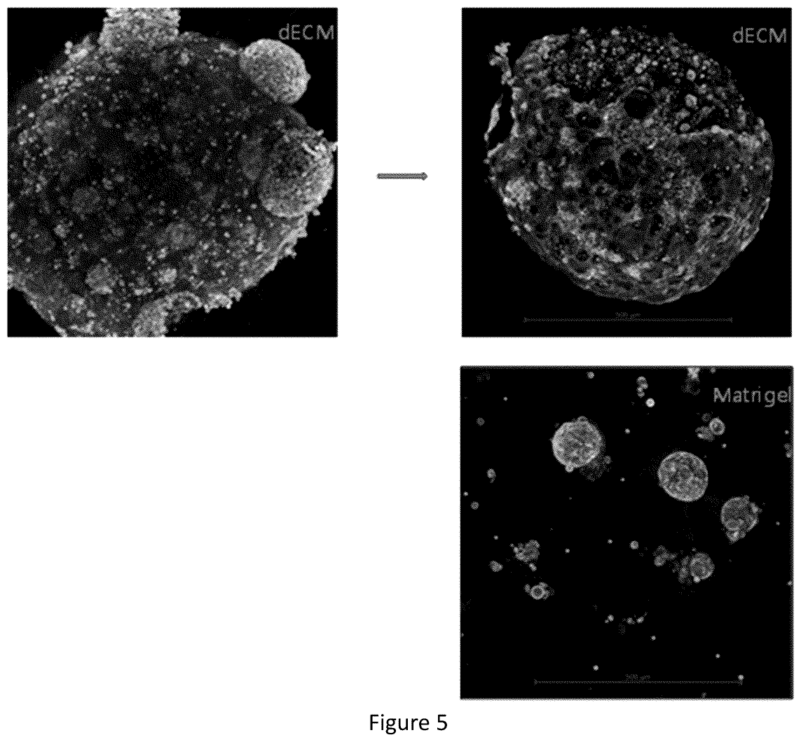

Specifically, as a result of observing the growth process of alveolar organoids cultured by dropping suspension culture using dECM, as shown in FIG. 5, it was observed that, as the organoids grow, they are fused with dECM and grow into one organoid, and as time passes, compared to culture in Matrigel in the late stage, several organoids appeared to gather together in the early stage, and there are differences in the number and size of organoids.

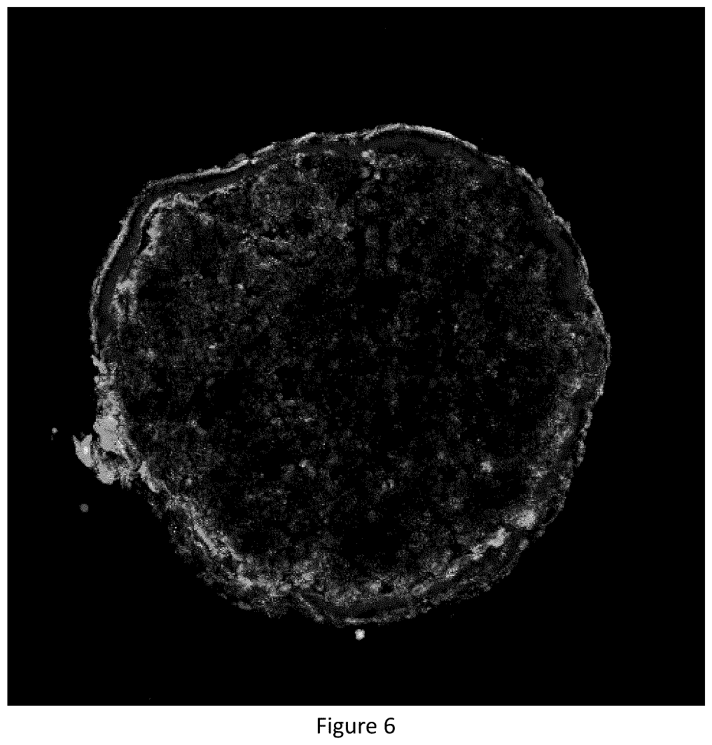

In addition, as a result of observing the cross-section of an organoid in which differentiation has been completed, as shown in FIG. 6, it was observed that the organoid maintained a similar morphology to actual lung tissue such that a large lumen is created inside the organoid, the morphology of type 1 alveolar cells, which are one of the main cells constituting the alveoli, forms the overall structure of the organoid, and type 2 alveolar cells forms partially present in the organoid.

### Example 3. Proteomics Analysis of Produced dECM

To confirm whether the components of the used dECM are suitable for organoid production, proteomics analysis was performed.

As shown in Table 2, since most of the components of the produced dECM consist of dECM proteins, it is expected to provide an environment suitable for growing organoids.

TABLE 2

| | |
|---|---|
| dECM | IF rod domain-containing protein |
| proteomics datas | LAMA5 protien |
| | Collagen alpha-3(VI) chain |
| | Collagen type VI alpha 2 chain |
| | Laminin subunit alpha-5 isoform X1 |
| | Thrombospondin 1 |
| | Laminin subunit beta-2 |
| | Trypsin |
| | Type VI collagen alpha-2 chain |
| | Laminin subunit beta 3 |
| | Matrilin 1 |
| | Matrilin 3 |
| | Complement C3 |
| | Basement membrane-specific heparan sulfate |
| | proteoglycan core protein isoform X4 |
| | Alpha2 chain of type I collagen |
| | Laminin subunit gamma 1 |
| | Type VI collagen alpha-1 chain |
| | Dermatopontin |

It should be understood by those of ordinary skill in the art that the above description of the present invention is exemplary, and the exemplary embodiments disclosed herein can be easily modified into other specific forms without changing the technical spirit or essential features of the present invention. Therefore, it should be interpreted that the exemplary embodiments described above are exemplary in all aspects and not restrictive.

1. A method of producing an alveolar organoid using a decellularized extracellular matrix (dECM), comprising:
(S1) preparing a bioink by mixing a first medium, type 2 alveolar cells, and dECM;
(S2) forming a cell culture droplet by dispensing the bioink into a plate;
(S3) gelating the cell culture droplet; and
(S4) isolating the gelated cell culture droplet from the plate and culturing it in a second medium.

2. The method of claim 1, wherein the method does not use Matrigel.

3. The method of claim 1, wherein the alveolar organoid is cultured in a non-adherent manner.

4. The method of claim 1, wherein the first medium is a small airway epithelial cell growth medium (SAGM).

5. The method of claim 1, wherein the second medium is an alveolarization medium.

6. The method of claim 5, wherein the alveolarization medium includes one or more selected from the group consisting of Ham's F12, dexamethasone, 3-isobutyl-1-methylxanthine (IBMX), B27 supplement, bovine serum albumin (BSA), hydroxyethyl piperazine ethane sulfonic acid (HEPES), calcium chloride, ITS premix, 8-Br-cAMP, fibroblast growth factor 7 (FGF7), and penicillin/streptomycin.

7. The method of claim 1, wherein the dECM is produced by the method as follows:
a) cutting lung tissue isolated from a subject to a size of 0.01 to 100 mm in diameter;
b) decellularizing the tissue cut in a);
c) removing fat from the decellularized tissue;
d) removing genetic material from the defatted tissue;
e) enzymatically digesting the genetic material-removed tissue with pepsin; and
f) neutralizing the enzymatically digested tissue.

8. The method of claim 7, wherein the decellularization is performed as follows:
b-1) treatment with sodium dodecyl sulfate (SDS);
b-2) treatment with Triton X-100; and
b-3) washing.

9. The method of claim 7, further comprising
g) performing gelation.

10. The method of claim 1, wherein the dECM comprises one or more selected from one or more selected from the group consisting of proteins listed in the following Table 2:
TABLE 2

| | |
|---|---|
| dECM | IF rod domain-containing protein |
| proteomics datas | LAMA5 protein |
| | Collagen alpha-3(VI) chain |
| | Collagen type VI alpha 2 chain |
| | Laminin subunit alpha-5 isoform X1 |
| | Thrombospondin 1 |
| | Laminin subunit beta-2 |
| | Trypsin |
| | Type VI collagen alpha-2 chain |
| | Laminin subunit beta 3 |
| | Matrilin 1 |
| | Matrilin 3 |
| | Complement C3 |
| | Basement membrane-specific heparan sulfate |
| | proteoglycan core protein isoform X4 |
| | Alpha2 chain of type I collagen |
| | Laminin subunit gamma 1 |
| | Type VI collagen alpha-1 chain |
| | Dermatopontin |

11. The method of claim 1, wherein the bioink includes dECM at a concentration of 0.01 to 10% (w/v).

12. An alveolar organoid prepared by the method of claim 1.

13. The alveolar organoid of claim 12, wherein the alveolar organoid is a form in which the dECM and alveolar cells are fused.

14. A method of treating a respiratory disease, comprising: administering a composition comprising the alveolar organoid of claim 12 to a subject in need thereof.
